(19) United States
(12) Patent Application Publication
(10) Pub. No.: US 2022/0364126 A1
(43) Pub. Date: Nov. 17, 2022

(54) Three-Vessel Reactor System for Producing Microbial Biosurfactants and Other Metabolites

(71) Applicant: Locus IP Company, LLC, Solon, OH (US)

(72) Inventors: Ken ALIBEK, Solon, OH (US); Nicholas CALLOW, Solon, OH (US); Tyler DIXON, Madison, OH (US); Sergey KRAVTSOV, Solon, OH (US)

(21) Appl. No.: 17/765,543

(22) PCT Filed: Nov. 2, 2020
(86) PCT No.: PCT/US2020/058490

§ 371 Date: Mar. 31, 2022

Related U.S. Application Data
(60) Provisional application No. 62/929,346, filed on Nov. 1, 2019.

(51) Int. Cl. C12P 1/04; C12M 1/00; C12M 1/02; C12M 1/06; C12M 1/34

(52) U.S. Cl. CPC C12P 1/04; C12M 23/58; C12M 41/18; C12M 29/18; C12M 29/08; C12M 27/06; C12M 29/20; C12M 41/26; C12M 41/04; C12R 2001/07

(57) ABSTRACT

The subject invention provides systems, apparatuses and methods for cultivating microorganisms and for producing microbial metabolites on a large scale. Specifically, in certain embodiments, a system comprising three separate, but connected, vessels is provided, wherein a first vessel serves as a feed tank for supplying nutrient medium to a second vessel, said second vessel serving as a submerged fermentation reactor; and wherein a third vessel serves as a collection container into which foam containing microbial growth by-products is transferred from the second vessel.

### CROSS-REFERENCE TO A RELATED APPLICATION

This application claims the benefit of U.S. Provisional Application Ser. No. 62/929,346, filed Nov. 1, 2019, which is incorporated herein by reference in its entirety.

### BACKGROUND OF THE INVENTION

Cultivation of microorganisms such as bacteria, yeasts and fungi is important for the production of a wide variety of useful bio-preparations. Microorganisms and their growth by-products play crucial roles in, for example, the food and beverage industry, pharmaceuticals, agriculture, mining, environmental remediation, and waste management. Notably, microbial production of biosurfactants is increasingly important for these and other industries.

Biosurfactants are a structurally diverse group of surface-active substances that are biodegradable, have low toxicity, and can be produced using selected organisms on renewable substrates. All biosurfactants are amphiphiles. They consist of two parts: a polar (hydrophilic) moiety and non-polar (hydrophobic) group. Due to their amphiphilic structure, biosurfactants increase the surface area of hydrophobic water-insoluble substances, increase the water bioavailability of such substances, and reduce the surface and interfacial tensions between the molecules of liquids, solids, and gases.

By reducing the interfacial tension between water and oil, biosurfactants help lower the hydrostatic pressure required to move entrapped liquid to overcome the capillary effect. Biosurfactants accumulate at interfaces, thus reducing interfacial tension and leading to the formation of aggregated micellar structures in solution. The formation of micelles provides a physical mechanism to mobilize, for example, oil in a moving aqueous phase. The ability of biosurfactants to form pores and destabilize biological membranes also permits their use as antibacterial, antifungal, and hemolytic agents to, for example, control pest and/or microbial growth.

There are multiple types of biosurfactants, which include low molecular weight glycolipids, lipopeptides, flavolipids and phospholipids, and high molecular weight polymers such as lipoproteins, lipopolysaccharide-protein complexes, and polysaccharide-protein-fatty acid complexes. The hydrocarbon chain of a fatty acid acts as the common lipophilic moiety of a biosurfactant molecule, whereas the hydrophilic part can be formed by, for example, esters, alcohols, carboxylates, amino acids, peptides and/or carbohydrates.

Lipopeptides, in particular, are oligopeptides synthesized by bacteria using large multi-enzyme complexes. They are frequently used as antibiotic compounds, and exhibit a wide antimicrobial spectrum of action, in addition to surfactant activities. All lipopeptides share a common cyclic structure consisting of a β-amino or β-hydroxy fatty acid integrated into a peptide moiety.

The most commonly studied family of lipopeptides, the surfactin family, consists of heptapeptides containing a β-hydroxy fatty acid with 13 to 15 carbon atoms. Surfactins are considered some of the most powerful biosurfactants. They are capable of some antiviral activity, as well as antifungal activity, and they exhibit strong synergy when used in combination with another lipopeptide, iturin A. Furthermore, surfactins may also be a key factor in the establishment of stable biofilms for the bacteria that produce them, while also inhibiting the biofilm formation of other bacteria, including Gram-negative bacteria.

Microbial biosurfactants are produced by a variety of microorganisms such as bacteria, fungi, and yeasts in response to the presence of a hydrocarbon source (e.g., oils, sugar, glycerol, etc.) in the growing media. Two principle forms of microbe cultivation exist: submerged cultivation and surface (or solid-state) cultivation. Bacteria, yeasts and fungi can all be grown using either the surface or submerged cultivation methods. The nutrient medium, which can either be in a liquid form (submerged) or a solid form (surface), typically includes a carbon source, a nitrogen source, salts and appropriate additional nutrients and microelements. The pH and oxygen levels are maintained at values suitable for a given microorganism.

Submerged and surface cultivation of microorganisms can be carried out at a small scale, e.g., in a test tube, to large scales, e.g., in a tank with a volume of thousands of gallons. However, with large-scale fermentation, particularly submerged fermentation, the concentration of desired microbial metabolites, such as biosurfactants, is often low with respect to the large volume of liquid in which they are produced.

One approach to obtaining higher concentrations of biosurfactants from submerged fermentation is to collect these molecules from foam that is produced during fermentation. Foam is a common by-product of fermentation, comprising a dispersion of gas in liquid, or more specifically, an agglomeration of gas bubbles separated from each other by a liquid film. This is because, for example, the presence of gas in the reactor (either as an input or output of the fermentation process) in combination with the components of the fermentation broth (which typically include a variety of foam-reinforcing proteins, peptides and/or starches) creates an ideal environment for foam formation and accumulation.

While foam production is favorable in the fermentation processes involved in, for example, beer brewing, other submerged fermentation processes can be seriously hindered by the production of foam. Without mitigation of foam production and accumulation, the foam can overflow into exhaust lines, reduce pump capacity, cause formation of deposits, clog filters and drains, increase cleaning time, and overall, cause loss of material and lower operating capacity.

Certain measures have been adopted to help control foam production in fermentation processes, including antifoam agents and mechanical foam breakers. Antifoam agents are typically chemical additives that prevent foam formation or break up foam that has already formed. These include, for example, insoluble oils, silicones, certain alcohols, stearates and glycols. While these additives are sometimes effective, they often require continuous replenishment in order to keep up with the rate of foam production. Furthermore, these additives can end up in the products of fermentation, thus requiring additional processing for removal of the antifoam agents.

There exists an enormous potential for the use of biosurfactants in a broad range of industries. However, current methods do not allow for the production of these products in the amounts needed for large scale applications, such as agriculture, and oil and gas recovery. Furthermore, systems and production methods are needed that reduce or eliminate the inefficiencies caused by foam production during fermentation.

### BRIEF SUMMARY OF THE INVENTION

The subject invention provides systems, apparatuses and methods for cultivating microorganisms and for producing microbial metabolites on a large scale. Specifically, the subject invention can be used for producing bacteria, yeasts and fungi, as well as growth by-products thereof, such as biosurfactants.

Embodiments of the present invention provide novel systems for continuous fermentation of microorganisms and production of growth by-products, as well as methods of fermentation using the same. More specifically, in certain embodiments, the present invention provides a system comprising three individual, but connected, vessels, wherein a first vessel serves as a feed vessel for supplying nutrients to a second vessel, which serves as a submerged fermentation reactor; and wherein a third vessel serves as a collection container into which foam containing microbial growth by-products is transferred from the second vessel.

In preferred embodiments, the three vessels function together as a single system. In some embodiments, the three-vessel system is controlled by a programmable logic controller (PLC). In certain embodiments, the PLC has a touch screen and/or an automated interface. The PLC can be used to start and stop the reactor system, and to monitor and adjust, for example, temperature, DO, pH, and nutrient feeding throughout fermentation. The three vessels can be made of, for example, plastic or another polymer, a metal, and/or a metal alloy.

In one embodiment, the system comprises a first vessel that serves as a feed tank for storing liquid nutrient medium. The first vessel supplies the nutrient medium to a second vessel, which serves as a submerged fermentation reactor. In preferred embodiments, the first and second vessels are tanks or columns. Even more preferably, the first and second vessels are stainless steel conical tanks, meaning they are cylindrical in shape with a cone-shaped bottom portion.

In one embodiment, each of the first and second vessels has a volume of about 1 gallon to about 500 gallons, or about 80 gallons to about 115 gallons. In one embodiment, the working volume of each of the first and second vessels is about 0.5 gallons to about 475 gallons, or about 75 gallons to about 90 gallons.

The first vessel can comprise a mixer, a temperature control system and/or a self-sterilization system. Additionally, in some embodiments, the first vessel can be connected to a water source so that water can be introduced directly into the first vessel for producing a liquid nutrient medium. In preferred embodiments, the water is filtered prior to introduction into the first vessel; thus, in some embodiments, the system comprises a water filter.

In some embodiments, the nutrient medium can be prepared in the first vessel or it can be pre-prepared and then a portion thereof is introduced into the first vessel. When prepared in the first vessel, raw nutrient medium ingredients are introduced into the vessel either before or after introduction of the filtered water, mixed, and stabilized at a temperature above, for example, 50° C., preferably around 80 to 100° C., for at least 60 minutes to ensure no contaminating microbial growth occurs within the first vessel.

In some embodiments, the temperature control system of the first vessel utilizes a shell-in-tube heat exchanger and recirculation pump that facilitates external circulation of liquid from the first vessel through the heat exchanger. This set-up reduces volume changes from steam condensate and provides uniform heating or cooling of the liquid nutrient medium. The temperature control system of the first vessel can be PLC-controlled to maintain the correct conditions for preventing microbial growth in the first vessel. In some embodiments, by continuously circulating the liquid nutrient medium in the first vessel, the temperature control system can also serve as a mixing system.

In one embodiment, the system comprises a second vessel, which serves as a submerged fermentation vessel. The first vessel is connected to the second vessel by a controlled pump, by a set of directional flow control valves, and/or by a metering device. In certain embodiments, the first vessel is connected to the second vessel by a set of directional flow control valves, which partition liquid nutrient medium being circulated out of the first vessel, through the heat exchanger, and back into the first vessel, and can be directed to deliver the liquid into the second vessel.

The second vessel can comprise its own (i.e., separate from the first vessel) means for mixing, temperature control and/or cleaning-in-place. Furthermore, the second vessel can comprise a set of floats or level sensors to detect liquid and/or foam levels within the vessel.

In certain embodiments, the system is further equipped with a multifunction pump, which is used for mixing/agitating the culture in the second vessel and supplying water and/or cleaning solutions to the clean-in-place means. The multifunction pump can also be connected to a second external heat exchanger for facilitating temperature control of the culture within the second vessel.

The second vessel can further comprise an aeration system capable of providing filtered air to the culture. The aeration system can, optionally, have an air filter for preventing contamination of the culture. The aeration system can function to keep the air level over the culture, the dissolved oxygen (DO), and the pressure inside the second vessel, at desired levels.

In certain embodiments, the second vessel can be equipped with a sparging system, through which the aeration system supplies air. Preferably, the sparging system comprises stainless steel injectors that produce microbubbles. In an exemplary embodiment, the spargers comprise a plurality of aerators comprising stainless steel microporous pipes (e.g., having tens or hundreds of holes that are 1 micron or less in size), which are connected to an air supply. The microporous design allows for proper dispersal of oxygen throughout the culture, while preventing contaminating microbes from entering the culture through the air supply.

In some embodiments, the clean-in-place means of the second vessel comprises a spray ball mounted to the side, top or bottom of the inside of the vessel for cleaning the vessel before and/or after fermentation cycles. The spray ball can be connected to the multifunction pump, which supplies water and/or cleaning solutions to the spray ball fixture.

The second vessel can be equipped with probes for monitoring fermentation parameters, such as, e.g., pH, temperature, DO and liquid/foam levels. The probes can be connected to a computer system, e.g., the PLC, which, based on readings from the probes, can automatically provide acid/base dosing, temperature control, aeration and/or can signal for a particular volume of liquid nutrient medium to be fed from the first vessel.

In some embodiments, the second vessel comprises an off-gas system for directing exhaust gas and foam produced during fermentation out of the second vessel. The foam and exhaust gas accumulate above the culture in the headspace of the second vessel, and are then transferred through the off-gas system into a third vessel (the collection vessel) via piping or tubing.

In certain embodiments, the third vessel is a tank or intermediate bulk container (IBC) made of plastic or stainless steel.

In some embodiments, the third vessel comprises an exhaust system. Upon collection of the foam and exhaust gas into the third vessel, the gas is released through the exhaust system, leaving behind the foam, which contains microbial growth by-products, such as biosurfactants.

In some embodiments, the third vessel comprises a recirculation system comprising a pump connected to tubing or piping for circulating, mixing and/or disrupting the foam.

In some embodiments, the third vessel comprises a pH probe for monitoring pH of the foam and signaling the PLC to dose acid/base solutions as needed to maintain a low pH. The foam containing the microbial growth by-product can be protected from possible biodegradation by reducing the pH to about 2.0 and circulating/mixing the foam through the recirculation system. A drain port allows for continuous or as-needed harvesting of the foam product for optional additional processing.

In preferred embodiments, the subject invention provides methods of cultivating microorganisms and/or producing microbial growth by-products without antifoam measures. In specific embodiments, the methods of cultivation are carried out in a system according to embodiments of the subject invention. In preferred embodiments, the growth by-products are biosurfactants.

In certain embodiments, the methods of cultivation comprise adding water and nutrients to a first vessel to produce a liquid nutrient medium; transferring all or a portion of the liquid nutrient medium to a second vessel; inoculating the second vessel with one or more microorganisms to produce a culture; cultivating the one or more microorganisms for an amount of time and under conditions favorable for production of one or more target growth by-products, wherein the one or more target growth by-products are produced into a foam that forms at the top of the culture; and directing the foam to a third vessel, where it is collected.

The foam can be drained from the third vessel at any point and processed further, for example, to extract and, optionally, purify the microbial growth by-product(s). In some embodiments, the method comprises mixing the foam continuously and/or mixing a pH adjuster with the foam while it is held in the third vessel in order to preserve the microbial growth by-product(s) from biodegradation.

In some embodiments, the methods are carried out continuously. In these embodiments, the fermentation process in the second vessel progresses, causing the liquid nutrient medium levels to drop as the medium is consumed and as foam is directed to the third vessel. Additional liquid nutrient medium is fed into the second vessel from the first vessel to refill and/or maintain the liquid levels in the second vessel. The first vessel can also be replenished with liquid nutrient medium as its supply is used up.

In one embodiment, the liquid nutrient medium comprises sources of, for example, carbon, nitrogen, proteins, vitamins and/or minerals. In one embodiment, the liquid nutrient medium comprises solid particulate anchoring carriers such as, for example, corn flour and/or cellulose grains, upon which microbes can attach and form biofilms. In certain embodiments, the nutrient medium is customized for production of desired biosurfactants and/or other metabolites.

In some embodiments, the microorganisms produced according to the subject methods are yeasts, bacteria or fungi. In specific embodiments, the microorganisms are bacteria.

In certain embodiments, the methods are used for co-cultivating two species of microbe, for example, Myxococcus xanthus and Bacillus amyloliquefaciens, wherein continuous co-cultivation using the subject three-vessel system results in enhanced production of biosurfactants by one or both of the microbes. In certain embodiments, the total cell biomass and/or the total production of biosurfactants achieved when using the subject methods is greater than when pure cultures of the individual microbes are cultivated on their own, and/or than when using non-continuous methods.

The collected foam can be processed by, for example, washing and/or centrifuging to extract the microbial growth by-product(s).

In specific embodiments, the microbial growth by-products produced according to the subject invention include lipopeptide biosurfactants, such as, e.g., surfactin, iturin, lichenysin, fengycin, plipastatins, kurstakins, arthrofactin, and/or viscosin. In certain embodiments, the methods can be used to produce, for example, foam comprising 5 g/L to 30 g/L of lipopeptides.

In certain embodiments, more than one type of biosurfactant is produced during co-cultivation, for example, glycolipids and/or fatty acid esters may be produced in addition to the lipopeptides. In some embodiments, the one or more growth by-products can also include other metabolites, for example, enzymes, biopolymers, acids, solvents, proteins, peptides, amino acids, alcohols, hormones, lipids, carbohydrates, antibiotics, other organic compounds and/or other bioactive compounds.

In certain embodiments, the subject invention provides microbe-based products produced according to the subject methods, as well as their uses in, for example, improved oil production, bioremediation and mining; waste disposal and treatment; promoting plant health and productivity; and reclaiming and/or restoring the health of soils.

The microbe-based products can comprise the entire culture produced according to the subject methods, including the microorganism(s) and/or their growth by-products, as well as residual growth medium, foam, particulate anchoring carrier and/or nutrients.

The microorganisms can be live, viable or in an inactive form. They can be in the form of a biofilm, vegetative cells, spores, and/or a combination thereof.

In preferred embodiments, no microbes are present, wherein the composition comprises microbial growth by-products, e.g., biosurfactants, that have been extracted from the foam and, optionally, purified.

Advantageously, operation of the subject systems can result in production of high concentrations of microbial growth by-products, such as biosurfactants, without the need for antifoam agents or mechanical foam breakers. Thus, the systems of the subject invention improve the efficiency of submerged fermentation of these microbe-based products by, for example, eliminating the need for purchasing and implementing antifoam measures, as well as eliminating the need for an extra step of processing the microbe-based products to remove antifoam agents.

### BRIEF DESCRIPTION OF THE DRAWINGS

FIG. 1 shows a front view drawing of a three-vessel system according to an embodiment of the subject invention.

### DETAILED DESCRIPTION OF THE INVENTION

The subject invention provides systems, apparatuses and methods for cultivating microorganisms and for producing microbial metabolites on a large scale. Specifically, the subject invention can be used for producing bacteria, yeasts and fungi, as well as growth by-products thereof, such as biosurfactants.

Embodiments of the present invention provide novel systems for continuous fermentation of microorganisms and production of growth by-products, as well as methods of fermentation using the same. More specifically, in certain embodiments, the present invention provides a system comprising three individual, but connected, vessels, wherein a first vessel serves as a feed vessel for supplying nutrients to a second vessel, which serves as a submerged fermentation reactor; and wherein a third vessel serves as a collection container into which foam containing microbial growth by-products is transferred from the second vessel.

In preferred embodiments, the subject invention provides methods of cultivating microorganisms and/or producing microbial growth by-products without antifoam measures. In specific embodiments, the methods of cultivation are carried out in a system according to embodiments of the subject invention. In preferred embodiments, the growth by-products are biosurfactants.

In certain embodiments, the subject invention provides microbe-based products produced according to the subject methods, as well as their uses in, for example, improved oil production, bioremediation and mining; waste disposal and treatment; promoting plant health and productivity; and reclaiming and/or restoring the health of soils.

Advantageously, operation of the subject systems can result in production of high concentrations of microbial growth by-products, such as biosurfactants, without the need for antifoam agents or mechanical foam breakers. Thus, the systems of the subject invention improve the efficiency of submerged fermentation of these microbe-based products by, for example, eliminating the need for purchasing and implementing antifoam measures, as well as eliminating the need for an extra step of processing the microbe-based products to remove antifoam agents.

### Selected Definitions

As used herein, a “biofilm” is a complex aggregate of microorganisms, such as bacteria, wherein the cells adhere to each other and/or to a surface using an extracellular polysaccharide matrix. The cells in biofilms are physiologically distinct from planktonic cells of the same organism, which are single cells that can float or swim in liquid medium.

As used herein, “co-cultivation” means cultivation of more than one strain or species of microorganism in a single fermentation system. In some instances, the microorganisms interact with one another, either antagonistically or symbiotically, resulting in a desired effect, e.g., a desired amount of cell biomass growth or a desired amount of metabolite production. In one embodiment, this antagonistic or symbiotic relationship can result in an enhanced effect, for example, the desired effect can be magnified when compared to what results from cultivating only one of the chosen microorganisms on its own. In an exemplary embodiment, one microorganism causes and/or stimulates the production of one or more metabolites by another microorganism, e.g., a Myxococcus sp. stimulates a Bacillus sp. to produce a biosurfactant.

As used herein, “enhancing” refers to improving and/or increasing. As used herein, “fermentation” refers to cultivation or growth of cells under controlled conditions. The growth could be aerobic or anaerobic. As used herein, an “isolated” or “purified” nucleic acid molecule, polynucleotide, polypeptide, protein, organic compound such as a small molecule (e.g., those described below), or other compound is substantially free of other compounds, such as cellular material, with which it is associated in nature. For example, a purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain is removed from the environment in which it exists in nature. Thus, the isolated strain may exist as, for example, a biologically pure culture, or as spores (or other forms of the strain), and in some embodiments, in association with a carrier.

In certain embodiments, purified compounds are at least 60% by weight the compound of interest. Preferably, the preparation is at least 75%, more preferably at least 85%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state or in spore form, or a mixture of both. The microbes may be planktonic or in a biofilm form, or a mixture of both. The by-products of growth may be, for example, metabolites (e.g., biosurfactants), cell membrane components, expressed proteins, and/or other cellular components. The microbes may be intact or lysed. The cells or spores may be totally absent, or present at, for example, a concentration of at least 1×10⁴, 1×10⁵, 1×10⁶, 1×10⁷, 1×10⁸, 1×10⁹, 1×10¹⁰, 1×10¹¹ or 1×10¹² or more CFU per milliliter of the composition.

The subject invention further provides “microbe-based products,” which are products that are to be applied in practice to achieve a desired result. The microbe-based product can be simply the microbe-based composition harvested from the microbe co-cultivation process. Alternatively, the microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “reduces” means a negative alteration of at least 1%, 5%, 10%, 25%, 50%, 75%, or 100%.

As used herein, “surfactant” means a compound that lowers the surface tension (or interfacial tension) between two liquids or between a liquid and a solid. Surfactants act as, e.g., detergents, wetting agents, emulsifiers, foaming agents, and/or dispersants. A “biosurfactant” is a surface-active substance produced by a living cell.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited components(s).

Unless specifically stated or obvious from context, as used herein, the term “or” is understood to be inclusive. Unless specifically stated or obvious from context, as used herein, the terms “a,” “and,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Fermentation System Design and Operation

Embodiments of the present invention provide novel systems for continuous fermentation of microorganisms and production of growth by-products, as well as methods of fermentation using the same. More specifically, in certain embodiments, the present invention provides a system comprising three individual, but connected, vessels, wherein a first vessel serves as a feed vessel for supplying nutrients to a second vessel, which serves as a submerged fermentation reactor; and wherein a third vessel serves as a collection container into which foam containing microbial growth by-products is transferred from the second vessel.

In preferred embodiments, the three vessels function together as a single system. The three vessels can be made of plastic or another polymer, a metal, and/or a metal alloy. In certain embodiments, each vessel comprises one or more ports through which fermentation materials and/or microorganisms can be introduced, sampled, and/or extracted.

In some embodiments, the three-vessel system is controlled by a programmable logic controller (PLC). In certain embodiments, the PLC has a touch screen and/or an automated interface. The PLC can be used to start and stop the reactor system, and to monitor and adjust, for example, temperature, DO, pH, and nutrient feeding throughout fermentation. In addition, each vessel may also have the capability for monitoring and controlling, for example, agitation, foaming, purity of microbial cultures, production of desired metabolites, and the like. The systems can further be adapted for remote monitoring of these parameters, for example with a tablet, smart phone, or other mobile computing device capable of sending and receiving data wirelessly.

In one embodiment, the system comprises a first vessel that serves as a feed tank for storing liquid nutrient medium. The first vessel supplies the nutrient medium to a second vessel, which serves as a submerged fermentation reactor. In preferred embodiments, the first and second vessels are tanks or columns. Even more preferably, the first and second vessels are stainless steel conical tanks, having a cylindrical body portion with a cone-shaped bottom portion or top portion.

In some embodiments, the cylindrical body portion of the first and second vessels have a height of about 5 ft. to about 25 ft., or about 8 ft. to about 15 ft. In some embodiments, the cone-shaped portion has a height of about 1 ft. to about 5 ft., or about 2 ft. to about 3 ft. In some embodiments, the diameter of the cylindrical body is about 1 ft. to about 5 ft., or about 1.5 ft. to about 3 ft.

In one embodiment, each of the first and second vessels has a volume of about 1 gallon to about 500 gallons, about 80 gallons to about 115 gallons, or about 88 gallons to about 110 gallons. In one embodiment, the working volume of each of the first and second vessels is about 0.5 gallon to about 475 gallons, about 75 gallons to about 90 gallons, or about 80 to about 85 gallons.

The first vessel can comprise a mixer, a temperature control system and/or a self-sterilization system. Additionally, in some embodiments, the first vessel can be connected to a water source so that water can be introduced directly into the first vessel for producing a liquid nutrient medium. In preferred embodiments, the water is filtered prior to introduction into the first vessel; thus, in some embodiments, the system comprises a water filter.

In some embodiments, the nutrient medium can be prepared in the first vessel or it can be pre-prepared and then introduced into the first vessel. When prepared in the first vessel, raw nutrient medium ingredients are introduced into the vessel either before or after introduction of the filtered water, mixed, and stabilized at a temperature above, for example, 50° C., preferably around 80 to 100° C., for at least 60 minutes to ensure no contaminating microbial growth occurs within the first vessel.

In one embodiment, the system comprises a second vessel, which serves as a submerged fermentation vessel. The first vessel is connected to the second vessel via piping or tubing, and flow of liquids through the piping or tubing is direct by a controlled pump, a set of directional flow control valves, and/or a metering device.

The temperature control system of the first vessel preferably comprises an out-flow tube or pipe extending from the bottom of the first vessel, a recirculation pump connected to said out-flow tube or pipe, a shell-in-tube heat exchanger having a first end and a second end, wherein the first end is connected to the recirculation pump, a first directional flow control valve fixed to the second end of the shell-in-tube heat exchanger, and an in-flow tube or pipe connected to the first directional flow control valve and extending into the first vessel at the top of the first vessel, wherein when the first directional flow control valve is open, the recirculation pump pumps liquid nutrient medium out of the first vessel through the out-flow tube or pipe, through the heat exchanger, through the first directional flow control valve, and back into the first vessel through the in-flow tube or pipe.

In certain embodiments, the system comprises a second directional flow control valve fixed to the second end of the heat exchanger along with the first directional flow control valve. The second directional flow control valve is connected to an in-flow tube or pipe extending into the second vessel.

When the first directional flow control valve is opened and the second flow control valve is closed, the liquid nutrient medium is pumped from the first vessel through the out-flow tube or pipe, circulates through the heat exchanger and through the first directional flow control valve, and then flows back into the first vessel through the first vessel in-flow tube or pipe.

When the second directional flow control valve is opened and the first flow control valve is closed, the liquid nutrient medium is pumped from the first vessel through the out-flow tube or pipe, circulates through the heat exchanger and through the first directional flow control valve, and then flows into the second vessel through the second vessel in-flow tube or pipe.

The second vessel can comprise its own (i.e., separate from the first vessel) means for mixing, temperature control and/or cleaning-in-place. Furthermore, the second vessel can comprise a set of floats or level sensors to detect liquid and/or foam levels within the vessel.

In certain embodiments, the system is further equipped with a multifunction pump, which is used for mixing/agitating the culture in the second vessel and supplying water and/or cleaning solutions to the clean-in-place means.

In some embodiments, the multifunction pump can be connected to external circulation piping or tubing, which is optionally connected to a second heat exchanger for facilitating temperature control of the culture within the second vessel. The multifunction pump circulates the culture out of the second vessel through the external piping or tubing, through the optional second heat exchanger, and back into the second vessel.

The system can also be adapted in other ways to ensure maintaining an appropriate fermentation temperature. For example, the outside of the vessels can be reflective to avoid raising the system temperature during the day if being operated outdoors. The system can also be insulated so the fermentation process can remain at appropriate temperatures in low temperature environments. Any of the insulating materials known in the art can be applied including fiberglass, silica aerogel, ceramic fiber insulation, etc. The insulation can surround any and/or all of the tubes and/or vessels of the system.

In one embodiment, the second vessel comprises a mixing motor, which is, preferably, located at the top of the second vessel. The motor is rotatably attached to a metal shaft that extends into the tank and is fixed with an impeller to help propel tank liquid from the top of the tank to the bottom of the tank and to ensure efficient mixing and gas dispersion throughout the culture. In one embodiment, the metal shaft with the impeller rotates on a slight diagonal axis (e.g., an axis at 15 to 60° from vertical).

In one embodiment, the impeller is a standard four-blade Rushton impeller. In one embodiment, the impeller comprises an axial flow aeration turbine and/or a small marine propeller. In one embodiment, the impeller design comprises customized blade shapes to produce increased turbulence.

The second vessel can further comprise an aeration system capable of providing filtered air to the culture. The aeration system can, optionally, have an air filter for preventing contamination of the culture. The aeration system can function to keep the air level over the culture, the dissolved oxygen (DO), and the pressure inside the second vessel, at desired (e.g., constant) levels.

In certain embodiments, the second vessel can be equipped with a sparging system, through which the aeration system supplies air in the form of air bubbles. If it is necessary to increase mass-exchange in the reactor, impellers of different types are used for additional breakage of bubbles and for intensifying mixing of the culture.

Preferably, the sparging system comprises stainless steel injectors that produce microbubbles. In an exemplary embodiment, the spargers comprise a plurality of aerators comprising stainless steel microporous pipes (e.g., having tens or hundreds of holes that are 1 micron or less in size), which are connected to an air supply. The microporous design allows for proper dispersal of oxygen throughout the culture, while preventing contaminating microbes from entering the culture through the air supply.

In some embodiments, the clean-in-place means of second vessel comprises a spray ball mounted to the side, top or bottom of the inside of the vessel for cleaning the vessel before and/or after fermentation cycles. The spray ball can be connected to the multifunction pump, which supplies water and/or cleaning solutions to the spray ball fixture.

The second vessel can be equipped with probes for monitoring fermentation parameters, such as, e.g., pH, temperature, DO and liquid/foam levels. The probes can be connected to the a computer system, e.g., the PLC, which, based on readings from the probes, can automatically provide acid/base dosing, temperature control, aeration and/or can signal for a particular volume of liquid nutrient medium to be fed from the first vessel.

In a further embodiment, the PLC may also be able to monitor the growth of microorganisms inside the second vessel (e.g., measurement of cell number and growth phases). Alternatively, a daily sample may be taken from the second vessel and subjected to enumeration by techniques known in the art, such as dilution plating technique.

In some embodiments, the second vessel comprises an off-gas system for directing exhaust gas and foam produced during fermentation out of the second vessel. The foam and exhaust gas accumulate above the culture in the headspace of the second vessel, and are then transferred through the off-gas system into a third collection vessel via piping or tubing.

In certain embodiments, the third vessel is a tank or intermediate bulk container (IBC) made of, for example, plastic or stainless steel.

In some embodiments, the third vessel comprises an exhaust system. Upon collection of the foam and exhaust gas into the third vessel, the gas is released through the exhaust system, leaving behind the foam, which contains microbial growth by-products, such as biosurfactants.

In some embodiments, the third vessel comprises a recirculation system comprising a pump connected to tubing or piping for circulating, mixing and disrupting the foam.

In some embodiments, the third vessel comprises means for monitoring and adjusting the pH of the collected foam. The pH monitoring and adjusting can be accomplished manually, for example, by sampling the foam through a port in the third vessel and testing its pH using tools known in the art.

The pH adjustment can also be accomplished by automatic means. The automatic pH adjustment can include a pH probe for monitoring pH of the foam and signaling the PLC to initiate dispensing of pH adjustment substances (i.e., acid or base solutions) appropriately to maintain a low pH. The pH can be set to a specific number by a user or can be pre-programmed to change the pH accordingly throughout operation of the system. In preferred embodiments, the pH is maintained at about 1.5 to about 2.5, or about 2.0.

Advantageously, the foam containing the microbial growth by-product is protected from possible biodegradation while sitting in the third vessel by maintaining this low pH, and also by circulating/mixing the foam. The third vessel can comprise a drain port, which allows for continuous or as-needed harvesting of the foam product from the third vessel for optional additional processing.

In one embodiment, the fermentation system is a mobile or portable bioreactor that may be provided for on-site production of a microbiological product including a suitable amount of a desired strain of microorganism. Because the microbiological product can be generated on-site of the application, without resort to the microbial stabilization, preservation, storage and transportation processes of conventional production, a much higher density of live microorganisms may be generated, thereby requiring a much smaller volume of the microbe-based composition for use in the on-site application.

The system can include a frame and/or a platform for supporting the system components. The system can include wheels for moving the system, as well as handles for steering, pushing and pulling when maneuvering the system.

The system can also be configured to be suitable for portability, e.g., via a trailer, a semi-trailer, a pickup truck bed, or a flatbed trailer.

### Microorganisms

The microorganisms grown according to the systems and methods of the subject invention can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, a single type of microbe is grown using the subject systems. In alternative embodiments, multiple microbes, which can be grown together without deleterious effects on growth or the resulting product, can be grown together There may be, for example, 2 to 3 or more different microbes grown in the second vessel at the same time.

In one embodiment, the beneficial microorganisms are yeasts and/or fungi. Yeast and fungus species suitable for use according to the current invention, include Acaulospora, Acremonium chrysogenum, Aspergillus, Aureobasidium (e.g., A. pullulans), Blakeslea, Candida (e.g., C. albicans, C. apicola, C. batistae, C. bombicola, C. floricola, C. kuoi, C. riodocensis, C. nodaensis, C. stellate), Cryptococcus, Debaryomyces (e.g., D. hansenii), Entomophthora, Hanseniaspora (e.g., H. uvarum), Hansenula, Issatchenkia, Kluyveromyces (e.g., K. phaffii), Lentinula spp. (e.g., L. edodes), Meyerozyma (e.g., M. guilliermondii), Monascus purpureus, Mortierella, Mucor (e.g., M. piriformis), Penicillium, Phythium, Phycomyces, Pichia (e.g., P. anomala, P. guilliermondii, P. occidentalis, P. kudriavzevii), Pleurotus (e.g., P. ostreatus P. ostreatus, P. sajorcaju, P. cystidiosus, P. cornucopiae, P. pulmonarius, P. tuberregium, P. citrinopileatus and P. flabellatus), Pseudozyma (e.g., P. aphidis), Rhizopus, Rhodotorula (e.g., R. bogoriensis); Saccharomyces (e.g., S. cerevisiae, S. boulardii, S. torula), Starmerella (e.g., S. bombicola), Torulopsis, Thraustochytrium, Trichoderma (e.g., T. reesei, T. harzianum, T. viride), Ustilago (e.g., U. maydis), Wickerhamiella (e.g., W. domericqiae), Wickerhamomyces (e.g., W. anomalus), Williopsis (e.g., W. mrakii), Zygosaccharomyces (e.g., Z. bailil), and others.

In certain embodiments, the microorganisms are bacteria, including Gram-positive and Gram-negative bacteria. The bacteria may be, for example Agrobacterium (e.g., A. radiobacter), Azotobacter (A. vinelandii, A. chroococcum), Azospirillum (e.g., A. brasiliensis), Bacillus (e.g., B. amyloliquefaciens, B. circulans, B. firmus, B. laterosporus, B. licheniformis, B. megaterium, Bacillus mucilaginosus, B. subtilis), Frateuria (e.g., F. aurantia), Microbacterium (e.g., M. laevaniformans), myxobacteria (e.g., Myxococcus xanthus, Stignatella aurantiaca, Sorangium cellulosum, Minicystis rosea), Pantoea (e.g., P. agglomerans), Pseudomonas (e.g., P. aeruginosa, P. chlororaphis subsp. aureofaciens (Kluyver), P. putida), Rhizobium spp., Rhodospirillum (e.g., R. rubrum), Sphingomonas (e.g., S. paucimobilis), and/or Thiobacillus thiooxidans (Acidothiobacillus thiooxidans).

In an exemplary embodiment, the microorganism is a myxobacteria. Myxobacteria are slime-forming, predatory bacteria that live in groups, or swarms. These swarms may form complex biofilms, as well as fruiting body structures, which are either simple or branched aggregates containing myxospores. During predation, the bacteria secrete predatory molecules, including enzymes, antibiotics and other secondary metabolites, which can include, for example, biosurfactants.

Myxobacteria include, for example, Myxococcus spp., Stignatella aurantiaca, Sorangium cellulosum, Minicystis rosea, and Chondromyces crocatus.

In certain preferred embodiments, the myxobacteria is a Myxococcus spp. bacterium selected from, for example, M. xanthus, M. fulvus, M. flavescens, M. macrosporus, M. stipitatus, M. virescens, M. coralloides, and M. disciformis. Even more preferably, the Myxococcus is M. xanthus.

In another exemplary embodiment, the microorganism is selected from Bacillus spp. bacteria. In general, Bacillus spp. bacteria are spore-forming, Gram-positive bacteria capable of producing valuable enzymes and biosurfactants. In preferred embodiments, the second microorganism is a Bacillus spp. such as, for example, B. amyloliquefaciens, B. coagulans, B. firmus, B. larvae, B. laterosporus, B lentimorbus, B. licheniformis, B. megaterium, B popilliae, B. polymyxa, B sphaericus, B. subtilis, and/or B. thuringiensis.

In yet another exemplary embodiment, two or more microorganisms are co-cultivated using the subject system, meaning they are grown together at the same time. For example, in one embodiment, M. xanthus and B. amyloliquefaciens are co-cultivated using the subject fermentation system. Advantageously, in some embodiments, the cell biomass from co-cultivation of these two microbes is greater than when pure cultures of the individual microbes are cultivated. Furthermore, in some embodiments, production of biosurfactants and/or other metabolites in co-culture is greater than when pure cultures of the individual microbes are used.

In certain embodiments, this enhanced production of growth by-products and/or metabolites is caused by the co-cultivation, wherein the presence of a competitor microorganism induces enhanced production of, for example, defensive molecules and/or self-growth promoters.

Other microbial strains including strains capable of accumulating significant amounts of, for example, glycolipid biosurfactants (e.g., rhamnolipids, mannosylerythritol lipids, cellobiose lipids and/or trehalose lipids), lipopeptide biosurfactants (e.g., surfactin, iturin, fengycin, arthrofactin and/or lichenysin), phospholipids (e.g., cardiolipin), fatty acid esters, mannoproteins, beta-glucan, and other metabolites that have bio-emulsifying and surface/interfacial tension-reducing properties, can be used in accordance with the subject invention.

### Methods of Cultivation Using the Subject Fermentation Systems

In preferred embodiments, the subject invention provides methods of cultivating microorganisms and/or producing microbial growth by-products without antifoam measures. In specific embodiments, the methods of cultivation are carried out in a three-vessel system according to embodiments of the subject invention. In preferred embodiments, the growth by-products are biosurfactants.

In general, the methods of cultivation comprise adding water and nutrients to a first vessel to produce a liquid nutrient medium; transferring a portion of the liquid nutrient medium to a second vessel; inoculating the second vessel with one or more microorganisms to produce a culture; cultivating the culture for an amount of time and under conditions favorable for production of one or more target growth by-products, wherein the one or more target growth by-products are produced into a foam that forms at the top of the culture; and directing the foam into a third vessel, where the foam is collected.

In one embodiment, the subject methods comprise introducing a liquid nutrient medium into a first vessel, which serves as a feed vessel. The components of the liquid nutrient medium can be pre-mixed prior to introduction into the first vessel, and/or they can be introduced individually into the first vessel and mixed therein.

In one embodiment, the liquid nutrient medium comprises a carbon source. The carbon source can be a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, rice bran oil, olive oil, corn oil, sesame oil, canola oil and/or linseed oil; powdered molasses, etc. These carbon sources may be used independently or in a combination of two or more.

In one embodiment, the liquid nutrient medium comprises a nitrogen source. The nitrogen source can be, for example, potassium nitrate, ammonium nitrate, ammonium sulfate, ammonium phosphate, ammonia, urea, and/or ammonium chloride. These nitrogen sources may be used independently or in a combination of two or more.

In one embodiment, one or more inorganic salts may also be included in the liquid nutrient medium. Inorganic salts can include, for example, potassium dihydrogen phosphate, monopotassium phosphate, dipotassium hydrogen phosphate, disodium hydrogen phosphate, potassium chloride, magnesium sulfate, magnesium chloride, iron sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These inorganic salts may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, proteins and microelements can be included, for example, peptone, yeast extract, potato extract, beef extract, soybean extract, banana peel extract, and the like, or in purified forms. Amino acids such as, for example, those useful for biosynthesis of proteins, can also be included.

The particulate anchoring carrier can be any material suitable for serving as a nucleation site for bacterial attachment and/or biofilm formation. In some embodiments, the material comprises a plurality of individual pieces, particles, and/or grains, that are about 0.1 μm to about 5 mm, about 0.2 μm to about 4 mm, about 0.3 μm to about 3 mm, about 0.4 μm to about 2 mm, about 0.5 μm to about 1 mm, or about 1 μm to about 0.5 mm in diameter. Bacteria will attach to the pieces and accumulate thereon, producing bacterial-carrier masses.

The anchoring carrier can be inert, or it can carry and/or comprise additional nutrients and/or microbial inoculant. In certain embodiments, the anchoring carrier can be porous. The anchoring carrier can comprise synthetic materials and/or naturally-derived materials.

In one embodiment, the anchoring carrier comprises sodium alginate beads. The beads can be prepared by, for example, continuously adding a solution comprising 1 to 5%, or 2 to 3% aseptic sodium alginate and, optionally, nutrients and/or bacterial inoculant, into a sterile 1 to 7%, or 2 to 5% calcium chloride solution to form beads.

In one embodiment, the anchoring carrier can comprise balls made of, for example, glass, a polymer (e.g., polylactic acid (PLA)), agar, or gelatin. In one embodiment, the anchoring carrier can be pieces of, for example, a chopped sponge or loofa. In one embodiment, the anchoring carrier can comprise, for example, whole, or pieces of, seeds, nuts, beans or even pieces of chopped fruit, such as bananas.

In preferred embodiments, the anchoring carrier comprises fine grains of cellulose (e.g., powdered cellulose) and/or corn flour (e.g., nixtamilized corn flour). In one embodiment, the use of fine grains (e.g., 0.1 μm to 5 mm) is preferred over larger particles because it facilitates scaling-up of the process.

Advantageously, the use of the anchoring carrier provides for increased production of bacterial biomass due to, for example, the increased surface area to which the bacteria can attach and accumulate. Additionally, the accumulation of bacterial biomass can lead to increases in the production of beneficial growth by-products, such as biosurfactants.

In one embodiment, prior to operation of the fermentation system, the components of the liquid nutrient medium can be sterilized. If used, the anchoring carrier is also preferably sterilized, for example, using an autoclave or other method known in the art. Additionally, water used for preparing the medium can be filtered to prevent contamination.

In one embodiment, sterilization of the liquid nutrient medium can be achieved by heating the components of the liquid culture medium to a temperature of about 85-100° C. In one embodiment, sterilization can be achieved by dissolving the components in 1% to 3% hydrogen peroxide in a ratio of 1:3 (w/v). In some embodiments, the method may further comprise adding additional acids and/or antimicrobials in the liquid nutrient medium for protecting the culture against contamination.

The methods can also comprise sterilizing or disinfecting the vessels and other components of the system prior to operation of the system. The vessels may be separated from, but connected to, a sterilizing unit, e.g., an autoclave. The cultivation equipment may also have a sterilizing unit that sterilizes in situ before starting the inoculation, e.g., by using a steamer. The air can be sterilized by methods know in the art. For example, air can pass through at least one filter before being supplemented into the system.

In certain embodiments, the subject methods comprise transferring the liquid nutrient medium into a second vessel, which serves as a submerged fermentation reactor. The second vessel is then inoculated with one or more microorganisms to produce a culture.

In one embodiment, the culture is cultivated in the second vessel for an amount of time and under conditions favorable for production of one or more target growth by-products by the microorganisms.

In some embodiments, the microbes grow in the form of a biofilm, for example, on the anchoring carrier. In some embodiments, the microbes grow in the liquid culture medium in planktonic form.

In certain embodiments, the second vessel is inoculated with two or more microorganisms, for example, a Myxococcus spp. bacterium and a Bacillus spp. bacterium. In an exemplary embodiment, the Myxococcus is M. xanthus and the Bacillus is B. amyloliquefaciens. Advantageously, in some embodiments, co-cultivation using the subject three-vessel system results in enhanced production of biosurfactants by one or both of the microbes. In certain embodiments, the total cell biomass and/or the total production of the one or more growth by-products achieved when using the subject methods can be greater compared to when pure cultures of the individual microbes are cultivated on their own.

In certain embodiments, the total cell biomass achieved according to the subject methods is at least 0.01%, 1%, 5%, 10%, 20%, 30%, 40%, 50%, 60%, 70%, 80%, 90%, 100%, or more, greater than when the two microorganisms are cultivated individually.

In certain embodiments, the total concentration of a growth by-product produced according to the subject methods is at least 0.01%, 1%, 5%, 10%, 20%, 30%, 40%, 50%, 60%, 70%, 80%, 90%, 100%, or more, greater than when the two microorganisms are cultivated individually.

The pH of the culture should be suitable for the microorganism(s) of interest. In some embodiments, the pH is about 2.0 to about 11.0, about 3.0 to about 10.0, about 4.0 to about 9.0, about 5.0 to about 8.0, or about 6.0 to about 7.0. In one embodiment, the pH is about 6.8. Buffers, and pH regulators, such as carbonates and phosphates, may be used to stabilize pH near a preferred value. When metal ions are present in high concentrations, use of a chelating agent in the liquid medium may be necessary.

In one embodiment, the method of cultivation is carried out at about 5° to about 100° C., about 15° to about 60° C., about 20° to about 45° C., or about 24° to about 30° C. In one embodiment, cultivation may be carried out continuously at a constant temperature. In another embodiment, cultivation may be subject to changing temperatures.

The method of cultivation can further provide oxygenation to the growing culture. One embodiment utilizes slow motion of air to remove low-oxygen containing air and introduce oxygenated air. The oxygenated air may be ambient air supplemented daily through mechanisms including air spargers for supplying bubbles of gas to the liquid. In certain embodiments, dissolved oxygen (DO) levels are maintained at about 25% to about 75%, about 30% to about 70%, about 35% to about 65%, about 40% to about 60%, or about 50% of air saturation. Air flow can be supplied at, for example, about 0.5 to about 2.0 v/m, or about 1.0 to about 1.5 vvm.

The microbial growth by-product(s) produced by the microorganism(s) may be retained in the microorganism(s) or secreted into the nutrient medium. In certain embodiments, the growth by-product(s) form a foam layer at the top of the culture, in the headspace of the second vessel.

The foam can be directed into a third vessel, either manually or using, for example, piping or tubing. In preferred embodiments, the foam is automatically directed out of the second vessel and into the third collection vessel through piping or tubing via the off-gas system.

Because, in certain embodiments, the foam contains microbial growth by-products that are sensitive to changes in pH, the third vessel may be fitted with a pH sensor to measure pH of the foam so that it can be adjusted if necessary. For example, in some embodiments, the desired pH range for the foam is about 1.5 to about 3.0, or about 2.5 to about 2.0.

In some embodiments, the method comprises mixing the foam in the third vessel and/or mixing a pH adjuster (an acid, base or buffer, e.g., HCl, KOH, NaOH, H₂SO₄, and/or H₃PO₄) with the foam while it is held in the third vessel in order to maintain the desired pH range and preserve the microbial growth by-product(s) from biodegradation.

In certain embodiments, the foam can be drained from the third vessel at any point. In some embodiments, the method further comprises extracting, concentrating and/or purifying the microbial growth by-product(s) from the foam. Alternatively, the microbial growth by-products can be utilized in their crude form, meaning the foam is utilized without any further processing.

According to the subject methods, the microorganism(s) can be cultivated in the second vessel for a time period sufficient to achieve a desired effect, e.g., production of a desired amount of cell biomass or a desired amount of one or more microbial growth by-products. The biomass content of the culture may be, for example, from 5 g/l to 180 g/l or more, or from 10 g/l to 150 g/l.

In some embodiments, fermentation occurs for 24 hours to 1 week or, preferably, longer. The methods can be performed in a batch, quasi-continuous, or continuous processes. In preferred embodiments, the process is continuous, with the potential to be carried out indefinitely as long as liquid nutrient medium is replenished in the first vessel, and favorable conditions for growth and production of microbial growth by-products are maintained within the second vessel.

In some embodiments, the methods are carried out continuously. In these embodiments, the fermentation process in the second vessel progresses, causing the liquid nutrient medium levels to drop as the medium is consumed and as foam is removed to the third vessel. Additional liquid nutrient medium is fed into the second vessel from the first vessel to refill and/or maintain the liquid levels in the second vessel. The first vessel can also be replenished with liquid nutrient medium as its supply is used up.

In certain embodiments, the liquid nutrient medium is fed into the second vessel continuously, e.g., at a consistent rate from the start of fermentation until fermentation is halted. In other embodiments, the nutrient medium is fed into the second vessel only when needed (e.g., when liquid and/or foam levels drop below a pre-determined point in the second vessel, when foam production and transfer into the third vessel slows or stops, or when sporulation percentage in the second vessel is measured at 20% or greater).

In one embodiment, the foam can be transferred from the second vessel into the third vessel on a consistent basis, meaning every 1 to 24 hours, every other day, or every 2 to 7 days. In another embodiment, the foam is transferred automatically upon reaching a certain volume, for example, upon reaching a pre-determined height within the second vessel, as determined by a float or level. The foam that is transferred can be a cell-free foam, and/or it can contain some cells,

Foam that is collected from the second vessel can be processed by, e.g., washing and/or centrifuging to extract the microbial growth by-products. Optionally, the growth by-products can then be stored, purified, and/or used directly in crude form.

In one embodiment, some or all of the anchoring carrier, if used, can be harvested from the culture and washed using a solvent, for example, low concentration (e.g., 1 to 2%) ethanol. The resulting liquid is then centrifuged to separate growth by-products and cell mass.

Advantageously, the method and equipment of the subject invention reduce the capital and labor costs of producing microorganisms and their metabolites on a large scale. Furthermore, the cultivation processes of the subject invention reduce or eliminate the need to concentrate organisms after completing cultivation. The subject invention provides a cultivation method that not only substantially increases the yield of microbial products per unit of nutrient medium but also simplifies production and facilitates portability.

### Microbial Growth By-Products

The methods of the subject invention can be used to produce compositions comprising one or more useful microbial growth by-products such as, for example, biosurfactants and/or other microbial metabolites.

In preferred embodiments, the growth by-products are one or more biosurfactants. Biosurfactants according to the subject invention can include, for example, glycolipids, lipopeptides, flavolipids, phospholipids, fatty acid esters, fatty acid ethers, lipoproteins, lipopolysaccharide-protein complexes, and/or polysaccharide-protein-fatty acid complexes.

In specific embodiments, the one or more biosurfactants comprise one or more lipopeptides, such as, e.g., surfactin, lichenysin, iturin, fengycin, plipastatin, arthrofactin, kurstakins, bacillomycin, mycosubtilin, glomosporin, amphisin, syringomycin and/or viscosin. In some embodiments, the biosurfactants are also useful and/or known as antibiotics. In certain embodiments, the methods can be used to produce a foam comprising about 1 to about 50 g/L of lipopeptides, about 5 to about 40 g/L, about 10 to about 30 g/L, or about 15 to about 20 g/L.

In some embodiments, the microorganisms can also produce one or more additional types of biosurfactants, such as glycolipids (e.g., rhamnolipids, sophorolipids, trehalose lipids, cellobiose lipids and/or mannosylerythritol lipids) and/or fatty acid esters (e.g., oleic fatty acid esters). In certain embodiments, the methods can be used to produce about 0.5 to about 10 g/L of the one or more additional types of biosurfactants, or about 1 to about 5 g/L.

In some embodiments, the microbial growth by-products include other metabolites. As used herein, a “metabolite” refers to any substance produced by metabolism (e.g., a growth by-product), or a substance necessary for taking part in a particular metabolic process, for example, enzymes, enzyme inhibitors, biopolymers, acids, solvents, gases, proteins, peptides, amino acids, alcohols, pigments, pheromones, hormones, lipids, ectotoxins, endotoxins, exotoxins, carbohydrates, antibiotics, anti-fungals, anti-virals and/or other bioactive compounds. The metabolite content produced by the method can be, for example, at least 0.1%, 1%, 5%, 10%, 20%, 30%, 40%, 50%, 60%, 70%, 80%, or 90%. In some embodiments, the metabolites are produced into the foam. In some embodiments, the metabolites are produced into the liquid culture.

Enzymes according to the subject invention can include, for example, oxidoreductases, transferases, hydrolases, lyases, isomerases and/or ligases. Specific types and/or subclasses of enzymes according to the subject invention can also include, but are not limited to, nitrogenases, proteases, amylases, glycosidases, cellulases, glucosidases, glucanases, galactosidases, moannosidases, sucrases, dextranases, hydrolases, methyltransferases, phosphorylases, dehydrogenases (e.g., glucose dehydrogenase, alcohol dehydrogenase), oxygenases (e.g., alkane oxygenases, methane monooxygenases, dioxygenases), hydroxylases (e.g., alkane hydroxylase), esterases, lipases, ligninases, mannanases, oxidases, laccases, tyrosinases, cytochrome P450 enzymes, peroxidases (e.g., chloroperoxidase and other haloperoxidases), and lactases.

In certain embodiments, the one or more growth by-products include antibiotic compounds, such as, for example, aminoglycosides, amylocyclicin, bacitracin, bacillaene, bacilysin, bacilysocin, corallopyronin A, difficidin, etnangien gramicidin, β-lactams, licheniformin, macrolactinsublancin, oxydifficidin, plantazolicin, ripostatin, spectinomycin, subtilin, tyrocidine, and/or zwittermicin A. In some embodiments, an antibiotic can also be a type of biosurfactant.

In certain embodiments, the one or more growth by-products include anti-fungal compounds, such as, for example, fengycin, surfactin, haliangicin, mycobacillin, mycosubtilin, and/or bacillomycin. In some embodiments, an anti-fungal can also be a type of biosurfactant.

In certain embodiments, the one or more growth by-products include other bioactive compounds, such as, for example, butanol, ethanol, acetate, ethyl acetate, lactate, acetoin, benzoic acid, 2,3-butanediol, beta-glucan, indole-3-acetic acid (IAA), lovastatin, aurachin, kanosamine, reseoflavin, terpentecin, pentalenolactone, thuringiensin (β-exotoxin), polyketides (PKs), terpenes, terpenoids, phenyl-propanoids, alkaloids, siderophores, as well as ribosomally and non-ribosomally synthesized peptides, to name a few.

### Microbe-Based Products

In certain embodiments, the subject invention provides microbe-based products produced according to the subject methods, as well as their uses in, for example, improved oil production, bioremediation and mining; waste disposal and treatment; promoting plant health and productivity; and reclaiming and/or restoring the health of soils.

The microbe-based products can comprise the entire culture produced according to the subject methods, including the microorganisms(s) and/or their growth by-products, as well as residual growth medium, foam, particulate anchoring carrier and/or nutrients.

The microorganisms can be live, viable or in an inactive form. They can be in the form of a biofilm, vegetative cells, spores, and/or a combination thereof.

In preferred embodiments, no microbes are present, wherein the composition comprises microbial growth by-products, e.g., biosurfactants, that have been extracted from the culture in the form of a foam, and, optionally, purified.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or foam resulting from the microbial growth can be removed from the system and transferred via, for example, piping for immediate use.

In other embodiments, the composition (microbes, broth and/or foam) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 gallon to 1,000 gallons or more. In certain embodiments the containers are 0.5 gallons, 2 gallons, 5 gallons, 25 gallons, or larger.

Upon harvesting the microbe-based composition from the system, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, pesticides, and other ingredients specific for an intended use.

Advantageously, in accordance with the subject invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, the product is stored at or below a temperature such as, for example, 20° C., 15° C., 10° C., 5° C. or 4° C., or less. If cells are present and in spore form, the product is, in one embodiment, stored and transported at a low temperature, not higher than 15° C., in order to prevent premature germination.

### Methods of Use

The compositions of the subject invention can be used for a variety of purposes. In one embodiment, the composition can be used in agriculture. For example, methods are provided wherein a composition produced according to the subject invention is applied to a plant and/or its environment to treat and/or prevent the spread of pests and/or diseases. The composition can also be useful for enhancing water dispersal and absorption in the soil, as well as enhance nutrient absorption from the soil through plant roots, facilitate plant health, increase yields, and manage soil aeration.

In one embodiment, the subject compositions can be highly advantageous in the context of the oil and gas industry. When applied to an oil well, wellbore, subterranean formation, or to equipment used for recovery oil and/or gas, the compositions produced according to the subject invention can be used in methods for enhancement of crude oil recovery; reduction of oil viscosity; removal and dispersal of paraffins and/or asphaltenes from rods, tubing, liners, and pumps; prevention of equipment corrosion; recovery of oil from oil sands and stripper wells; enhancement of fracking operations as fracturing fluids; reduction of H₂S concentration in formations and crude oil; and cleaning of tanks, flowlines and pipelines.

In one embodiment, the compositions produced according to the subject invention can be used to improve one or more properties of oil. For example, methods are provided wherein the composition is applied to oil or to an oil-bearing formation in order to reduce the viscosity of the oil, convert the oil from sour to sweet oil, and/or to upgrade the oil from heavy crude into lighter fractions.

In one embodiment, the compositions produced according to the subject invention can be used to clean industrial equipment. For example, methods are provided wherein a composition is applied to oil production equipment such as an oil well rod, tubing and/or casing, to remove heavy hydrocarbons, paraffins, asphaltenes, scales and other contaminants from the equipment. The composition can also be applied to equipment used in other industries, for example, food processing and preparation, agriculture, paper milling, and others where fats, oils and greases build up and contaminate and/or foul the equipment.

In one embodiment, the compositions produced according to the subject invention can be used to enhance animal health. For example, methods are provided wherein the composition can be applied to animal feed or water, or mixed with the feed or water, and used to prevent the spread of disease in livestock and aquaculture operations, reduce the need for antibiotic use in large quantities, as well as to provide supplemental proteins and other nutrients.

In one embodiment, the compositions produced according to the subject invention can be used to prevent spoilage of food, prolong the consumable life of food, and/or to prevent food-borne illnesses. For example, methods are provided wherein the composition is applied to a food product, such as fresh produce, baked goods, meats, and post-harvest grains, to prevent undesirable microbial growth.

Other uses for the subject compositions include, but are not limited to, biofertilizers, biopesticides, bioleaching, bioremediation of soil and water, pharmaceutical adjuvants (for increasing bioavailability of orally ingested drugs), cosmetic products, control of unwanted microbial growth, and many others.

### Local Production of Microbe-Based Products

In preferred embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The distributed microbe growth facilities can be located at the location where the microbe-based product will be used. For example, the microbe growth facility may be less than 300, 250, 200, 150, 100, 75, 50, 25, 15, 10, 5, 3, or 1 mile from the location of use.

The microbe growth facilities of the subject invention produces fresh, microbe-based compositions, comprising the microbes themselves, microbial metabolites, and/or other components of the broth in which the microbes are grown. If desired, the compositions can have a high density of vegetative cells or propagules, or a mixture of vegetative cells and propagules.

Because the microbe-based product is generated locally, without resort to the microorganism stabilization, preservation, storage and transportation processes of conventional microbial production, a much higher density of bacteria cells and/or propagules can be generated, thereby requiring a smaller volume of the microbe-based product for use in the on-site application or which allows much higher density microbial applications where necessary to achieve the desired efficacy. Local generation of the microbe-based product also facilitates the inclusion of the growth broth in the product. The broth can contain agents produced during the fermentation that are particularly well-suited for local use.

Advantageously, the compositions can be tailored for use at a specified location. The microbe growth facilities provide manufacturing versatility by the ability to tailor the microbe-based products to improve synergies with destination geographies and harness the power of naturally-occurring local microorganisms and their metabolic by-products to improve oil production. Local microbes can be identified based on, for example, salt tolerance and ability to grow at high temperatures.

Advantageously, these microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated broth and metabolites in which the cells are originally grown.

The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Three-Vessel Bioreactor

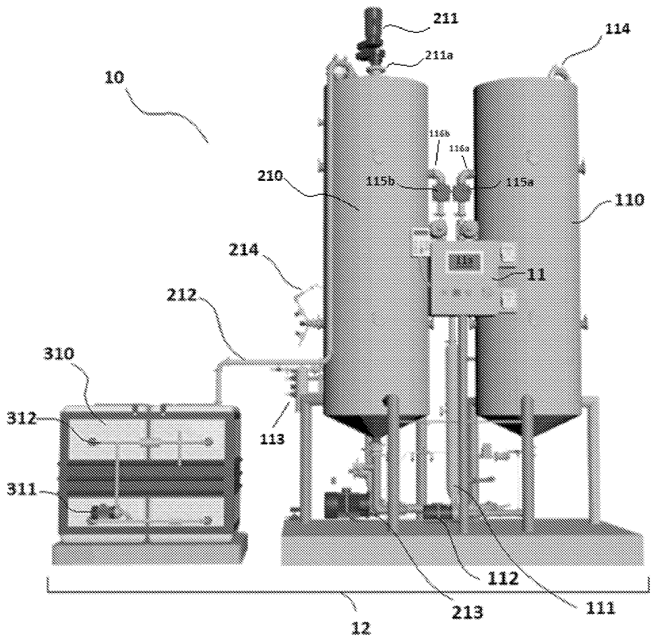

FIG. 1 shows a front view of a three-vessel system 10 according to one embodiment of the subject invention. The system 10 is situated on a level platform 12. The system 10 is controlled by a programmable logic controller (PLC) 11 with a touch screen and/or an automated interface 11a. The PLC 11 can be used to start and stop the reactor system 10, and to monitor and adjust, for example, temperature, DO, pH, and nutrient feeding throughout fermentation.

The system 10 comprises a first vessel 110 that serves as a feed tank for storing liquid nutrient medium. The first vessel 110 supplies the nutrient medium to a second vessel 210, which serves as a submerged fermentation reactor. The first 110 and second 210 vessels are stainless steel conical tanks having a cylindrical body portion with a cone-shaped bottom portion.

Each of the first 110 and second 210 vessels have a volume of about 110-115 gallons, and a working volume of about 80-90 gallons.

The first vessel 110 is connected to a water source 113 via piping 114 so that water can be introduced directly into the first vessel 110 for producing a liquid nutrient medium. The first vessel 110 can also comprise a water filter (not shown) for preventing contamination.

The temperature control system of the first vessel 110 utilizes a shell-in-tube heat exchanger 111 and recirculation pump 112 that facilitate external circulation of liquid from the first vessel 110 through out-flow piping (not shown), through the heat exchanger 111, through a first directional flow control valve 115a fixed to the heat exchanger 111, and through an in-flow pipe 116a back into the first vessel 110. This set-up reduces volume changes from steam condensate and provides uniform heating or cooling of the liquid nutrient medium. The temperature control system can be PLC-controlled to maintain the correct temperature for preventing microbial growth in the first vessel 110.

The system 10 further comprises a second vessel 210, which serves as a submerged fermentation vessel. The first vessel 110 is connected to the second vessel 210 via the shell-in-tube heat exchanger 111 and recirculation pump 112. A second directional flow control valve 115b is also fixed to the heat exchanger 111, and is connected to in-flow piping 116b that extends into the second vessel 210. The PLC 11 controls whether the first directional flow control valve 115a or the second directional flow control valve 115b is open, thereby controlling whether the liquid nutrient medium is circulated, respectively, out of the first vessel 110 and back into the first vessel 110, or out of the first vessel 110 and into the second vessel 210.

The second vessel 210 comprises ports 214 for, e.g., inoculating the vessel 210 with one or more microorganisms, probing/testing fermentation parameters, manually introducing pH modulators, antimicrobials, and/or additional nutrients, as well as sampling the culture.

The second vessel 210 also comprises a mixing apparatus, which comprises a mixing motor 211 located at the top of the second vessel 210. The motor 211 is rotatably attached to a metal shaft 211a that extends into the vessel 210 and is fixed with an impeller (not shown) to help propel liquid from the top of the vessel 210 to the bottom and to ensure efficient mixing and gas dispersion throughout the culture.

Additionally, the second vessel 210 comprises probes (not shown) for monitoring temperature, pH and/or dissolved oxygen (DO) levels of the culture being produced therein. The probes are connected to the PLC 11 and transmit measurements thereto for automatic modulation of the fermentation parameters. Furthermore, liquid levels in the second vessel 210 are monitored by floats or level sensors (not shown) to detect liquid and/or foam levels within the vessel 210 and signal to the PLC 11 to switch the directional flow control valves to direct liquid nutrient medium into the second vessel 210.

The second vessel 210 further comprises an aeration system (not shown) capable of providing filtered air to the culture. The aeration system can, optionally, have an air filter for preventing contamination of the culture.

On the inside (not shown) of the second vessel 210 a sparging system is used to disperse microbubbles of air provided by the aeration system. Additionally, a clean-in-place apparatus comprising a stainless steel spray ball is mounted on the side, top or bottom of the inside of the vessel 210, which is used for cleaning the vessel 210 before and/or after fermentation cycles.

The system 10 is further equipped with a multifunction pump 213, which is used for mixing/agitating the culture in the second vessel 210 and supplying water and/or cleaning solutions to the clean-in-place means (not shown). The multifunction pump 213 can also be connected to a second external heat exchanger (not shown) for facilitating temperature control of the culture in the second vessel 210.

The second vessel 210 comprises an off-gas system (not shown) for directing exhaust gas and foam produced during fermentation out of the second vessel 210. The foam and exhaust gas accumulate above the culture in the headspace of the second vessel 210, and are then transferred through the off-gas system into a third collection vessel 310 via piping 212.

The third vessel 310 can be a tank, barrel or IBC made of plastic, another polymer, a metal or a metal alloy.

The third vessel 310 comprises an exhaust system (not shown). Upon collection of the foam and exhaust gas into the third vessel 310, the gas is released through the exhaust system, leaving behind the foam, which contains microbial growth by-products, such as biosurfactants.

The third vessel 310 comprises a recirculation pump 311 with external tubing 312 for mixing and disrupting the foam. The third vessel 310 comprises a pH probe (not shown) for monitoring pH of the foam and signaling the PLC 11 to dose acid/base solutions as needed to maintain a low pH. A drain port (not shown) allows for continuous or as-needed harvesting of the foam product from the third vessel 310 for optional additional processing.

### Example 2—Continuous Production of Lipopeptides

Referring to FIG. 1, a liquid nutrient medium is mixed with water in the first vessel 110 of the three-vessel system 10. The nutrient medium comprises:

| | |
|---|---|
| Glucose | 1 g/L to 5 g/L |
| Casein peptone | 1 g/L to 10 g/L |
| K₂HPO₄ | 0.01 g/L to 1.0 g/L |
| KH₂PO₄ | 0.01 g/L to 1.0 g/L |
| MgSO₄•7H₂O | 0.01 g/L to 1.0 g/L |
| NaCl | 0.01 g/L to 1.0 g/L |
| CaCO₃ | 0.5 g/L to 5 g/L |
| Ca(NO₃)₂ | 0.01 g/L to 1.0 g/L |
| Yeast extract | 0.01 g/L to 5 g/L |
| MnCl₂•4H₂O | 0.001 g/L to 0.5 g/L |
| Teknova trace element | 0.5 ml/L to 5 ml/L |

Fine grain particulate anchoring carrier is also suspended in the liquid nutrient medium. The carrier comprises cellulose (1.0 to 5.0 g/L) and/or corn flour (1.0 to 8.0 g/L).

Bacillus amyloliquefaciens inoculum is grown in a small-scale reactor for 24 to 48 hours. Myxococcus xanthus inoculum is grown in a 2 L working volume seed culture flask for 48 to 120 hours. The second vessel 210 is inoculated with both microorganisms.

pH in the second vessel 210 is maintained at about 6.8; temperature is maintained at about 24° C.; DO is maintained at about 50% of air saturation; and air flow rate is maintained at about 1 vvm.

A foam layer comprising microbial growth by-products is produced during fermentation and is purged out into the third vessel 310. A pH meter is used to monitor the pH of the foam: if the pH varies outside of the range of 2.0 to 3.0, pH adjusters are added to bring the pH back within that range for long-term preservation of the lipopeptides therein. Foam continues to be produced, purged from the second vessel 210, and collected in the third vessel 310 for 7 days or longer (e.g., indefinitely).

Sampling of the second vessel 210 and the third vessel 310 for CFU count, sporulation percentage and/or purity is performed at 0 hr., then twice per day throughout fermentation. Sampling can also occur at the time that foam is purged and collected. When/if sporulation percentage of the bacterial culture in the second vessel 210 is detected (using microscope slide estimation) to be greater than 20%, additional liquid nutrient medium is transferred from the first vessel 110 into the second vessel 210.

LC-MS analysis is carried out on acidified lipopeptide samples from the foam collection tank. The samples are stored at about 4° C.

The fermentation cycle is continued for at least one week, with nutrient medium feeding and foam collection occurring until, for example, foam can no longer be extracted from the fermenter.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

1. A system comprising a first vessel, a second vessel and a third vessel, wherein
the first vessel serves as a feed tank for a liquid nutrient medium;
the second vessel serves as a submerged fermentation reactor for one or more microorganisms; and
the third vessel serves as a collection container for foam produced in the second vessel during the submerged fermentation of the one or more microorganisms,
wherein the first vessel is connected to the second vessel, and the second vessel is connected to the third vessel, and
wherein the system is controlled by a programmable logic controller (PLC).

2. The system of claim 1, wherein the first vessel is connected to a water source for introducing water into the first vessel, and wherein the system further comprises a water filter through which the water passes prior to introduction into the first vessel.

3. The system of claim 1, wherein the first vessel comprises a temperature control system, said temperature control system comprising
an out-flow tube or pipe extending from the bottom of the first vessel,
a recirculation pump connected to said out-flow tube or pipe,
a shell-in-tube heat exchanger having a first end and a second end, wherein the first end is connected to the recirculation pump,
a first directional flow control valve fixed to the second end of the shell-in-tube heat exchanger, and
an in-flow tube or pipe connected to the first directional flow control valve and extending into the first vessel at the top of the first vessel,
wherein when the first directional flow control valve is open, the recirculation pump pumps liquid nutrient medium out of the first vessel through the out-flow tube or pipe, through the heat exchanger, through the first directional flow control valve, and back into the first vessel through the in-flow tube or pipe.

4. The system of claim 3, wherein the temperature control system of the first vessel maintains a temperature in the first vessel that is suitable for controlling microbial growth in the liquid nutrient medium while also mixing the liquid nutrient medium.

5. The system of claim 3, comprising a second directional flow control valve fixed to the second end of the heat exchanger along with the first directional flow control valve, and further comprises an in-flow tube or pipe extending into the second vessel.

6-7. (canceled)

8. The system of claim 1, wherein the second vessel comprises a mixing apparatus, an aeration system, a multifunction pump, a clean-in-place system, and an off-gas system; wherein the aeration system comprises a sparging system to inject microbubbles of air into the second vessel.

9. The system of claim 8, wherein the mixing apparatus comprises a mixing motor that is rotatably attached to a metal shaft fixed with an impeller, said metal shaft extending into the second vessel from the top of the second vessel.

10. (canceled)

11. The system of claim 8, wherein the multifunction pump is connected to a second shell-in-tube heat exchanger, and wherein the multifunction pump circulates the culture from the second vessel through the heat exchanger and back into the second vessel, thereby mixing the culture and facilitating temperature control of the culture.

12. (canceled)

13. The system of claim 8, wherein foam and exhaust gas from the second vessel are directed into the third vessel through tubing and/or piping via the off-gas system.

14. The system of claim 1, wherein the third vessel comprise an exhaust system for releasing exhaust gas collected from the second vessel.

15. The system of claim 1, wherein the third vessel comprises a recirculation pump connected to external tubing for mixing and disrupting the foam.

16. The system of claim 1, wherein the third vessel comprises a pH probe connected to the PLC for monitoring the pH of the foam, and wherein the PLC instructs administration of pH adjusting solutions into the foam when pH levels above 2.0 are detected.

17. A method of producing a microorganism and/or a growth by-product thereof, the method comprising
introducing water and nutrients into a first vessel to produce a liquid nutrient medium, said first vessel serving as a feed tank;
transferring a portion of the liquid nutrient medium from the first vessel to a second vessel, said second vessel serving as a submerged fermentation reactor;
inoculating the second vessel with the microorganism to produce a culture;
cultivating the culture for an amount of time and under conditions favorable for production of one or more target growth by-products, wherein the one or more growth by-products are produced into a foam that forms at the top of the culture; and
directing the foam into a third vessel, where the foam is collected.

18-19. (canceled)

20. The method of claim 17, wherein the microorganism is a Myxococcus spp. bacterium.

21. (canceled)

22. The method of claim 17, wherein the microorganism is a Bacillus spp. bacterium.

23. The method of claim 22, wherein the Bacillus is B. amyloliquefaciens.

24. The method of claim 17, wherein the second vessel is inoculated with two or more microorganisms at the same time.

25. The method of claim 24, wherein the two or more microorganisms are M. xanthus and B. amyloliquefaciens.

26. The method of claim 17, wherein the growth by-product is a biosurfactant.

27-29. (canceled)

30. The method of claim 17, further comprising continuously mixing the foam in the third vessel.

31. The method of claim 17, further comprising testing the pH of the foam in the third vessel, and if the pH is 2.0 or higher, mixing a pH adjusting solution with the foam to reduce the pH to 2.0 or lower.

32-33. (canceled)

34. The method of claim 17, which is carried out continuously for 1 week or longer, wherein the culture in the second vessel is continually replenished with liquid nutrient medium from the first vessel, and wherein the foam is continually transferred from the second vessel into the third vessel.
